(19) United States
(12) Patent Application Publication
(10) Pub. No.: US 2026/0060534 A1
(43) Pub. Date: Mar. 5, 2026

(54) DEVICE FOR TESTING VISION THROUGH AN INTRAOCULAR LENS

(71) Applicants: ACMIT GMBH, Wiener Neustadt (AT); QMP HOLDING GMBH, Mannheim (DE)

(72) Inventors: Wolfgang BREZNA, Wiener Neustadt (AT); Nikolaus DRAGOSTINOFF, Wien (AT); Martin KORNFELD, Wien (AT); Olivier LA SCHIAZZA, Heidelberg (DE); Kirsten LUX, Mödling (AT); Nicole PLANK, Forchtenstein (AT)

(21) Appl. No.: 19/106,710

(22) PCT Filed: Aug. 1, 2023
(86) PCT No.: PCT/EP2023/071266

§ 371 Date: Feb. 26, 2025

(30) Foreign Application Priority Data
Aug. 30, 2022 (EP) 22192915.1

(51) Int. Cl. A61B 3/04; A61B 3/103

(52) U.S. Cl. CPC A61B 3/04; A61B 3/103

(57) ABSTRACT

A device for testing vision through an intraocular lens has a support, which supports a corneal optic, a mounting for the intraocular lens, and an ocular optic one after the other distally to proximally in a central viewing axis. The ocular optic includes, distally, a correcting lens and, proximally thereto, a diverging lens and a collecting lens, wherein the correcting lens has a distal concave lens surface and a proximal convex lens surface.

The present invention relates to a device for testing vision through an intraocular lens, with a support which supports a corneal optic, a mounting for the intraocular lens, and an ocular optic one after the other distally to proximally in a central viewing axis.

The implementation of a phakic, aphakic or pseudophakic intraocular lens (“intraocular lens”, IOL) in a patient's eye is an extremely critical process. The IOL must be adapted as well as possible to the individual requirements of the patient, since every subsequent replacement carries with it an unnecessary operating risk. Thus, it is necessary and desirable to thoroughly examine the patient's vision through an IOL to be implanted, i.e. its optical properties, even before an implementation, in order to avoid a replacement in vivo at all events.

Either computer simulations or optical testing devices, into which the IOL to be implanted is inserted, are conventionally used for this purpose. However, images produced by means of computer simulation with the aid of an eye with average anatomy, which are shown to the patient, almost always reproduce only very general properties of the IOL. Using simulations, therefore, it is impossible to predict reliably the patient's subjective visual impression through the IOL selected for the implantation. Moreover, it is especially difficult to reliably simulate different viewing situations, e.g. different lighting conditions and visual ranges such as distant vision when viewing a landscape and near vision when reading a book.

Optical testing devices are better suited for testing the subjective visual impression in different situations. The testing devices known to date, however, are very limited in the field of view and are not therefore capable of testing a wide range of the patient's viewing angles.

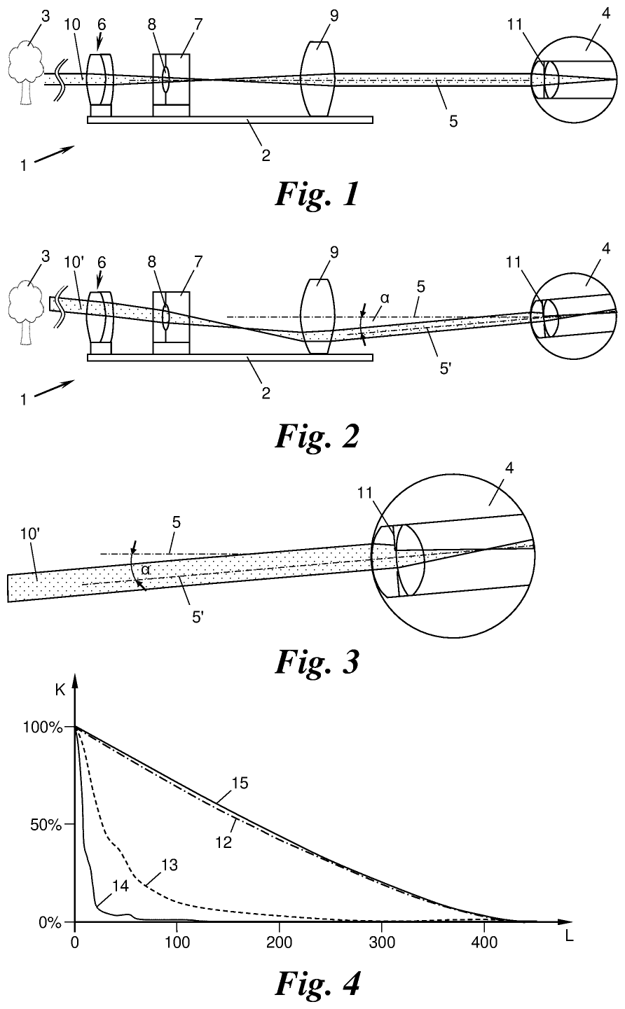

FIG. 1 to 3 show one such conventional optical testing device 1 with a support 2, which supports a corneal optic 6 (also referred to as a corneal model optic), a mounting 7 for IOL 8 and a collecting lens 9 distally (close to the observed surrounding 3) to proximally (close to the patient's eye 4) along a central viewing axis 5. If, by a rotation of eye 4, a viewing axis 5′ tilted at a viewing angle α is assumed (FIG. 2), a light beam 10′ passing through device 1—and therefore the perceivable image—is increasingly cut off by iris 11 of eye 4 (FIG. 3). Moreover, the remaining actually perceived image, on account of defocusing, has a contrast and a resolution which differ significantly from the contrast and resolution of IOL 8 in the actually implanted state—referred to hereinafter as “implanted IOL”, as can be seen from the modulation transfer function represented in FIG. 4 (“modulation transfer function”, MTL) of device 1 for the viewing angle α=0° (dot-dashed line 12), α=2.5° (dashed line 13) and α=5° (dotted line 14) on the one hand and the nominal MTF of implanted IOL 8 (continuous line 15) on the other hand.

For all these reasons, it is still difficult for the patient to select from the wide variety of versions of IOLs, be they monofocal, bifocal, multifocal, EDOF (“Extended Depth of Focus”) lenses, light-adjustable lenses (LAL) etc., the IOL which guarantees the optimum subjective visual impression.

The aim of the invention is to create a device for testing the vision through an IOL, which overcomes the drawbacks of the prior art and enables lens testing with an extended field of view.

This aim is achieved with a device of the type mentioned at the outset, which is characterised according to the invention by the fact that the ocular optic comprises distally a correcting lens and, proximally thereto, a diverging lens and collecting lens, wherein the correcting lens has a distal concave lens surface and a proximal convex lens surface.

The combination according to the invention of a correcting lens, a diverging lens and a collecting lens in the ocular optic permits, through the special shape of the lens surfaces, the field of view of the device or of a patient's eye looking through the latter to be extended and at the same time defocusing and contrast errors in the extended field of view to be corrected and ideally to be eliminated.

Thus, through the inventive interaction of the concave-convex correcting lens and the downstream diverging and collecting lenses, different vergences and different angular differences are produced between the respective entry beam and exit beam in respect of the central viewing axis or optical axis for different angles of incidence of light rays. Precisely these special vergences or angular differences permit—in combination with the corneal optic—incident light beams from respective spatial angles to be directed essentially onto the same point of the eye for different viewing angles and thus a viewing angle correction, i.e. a focussed view through the IOL agreeing in resolution and contrast with the implanted IOL to be obtained for different viewing angles.

The corneal optic can—in a particularly efficient variant—model an average cornea, e.g. according to the Liou-Brennan Eye Model, in order to carry out a rapid vision test; or—in a particularly precise variant—can model the cornea of the individual patient, e.g. after its prior measurement, in order to enable testing of the IOL that is as realistic as possible for the individual patient.

Not least, the device according to the invention enables testing of the subjective visual impression through the IOL in different situations, whether it is the testing of distant vision and near vision. The device according to the invention is thus particularly well suited for the testing of a wide variety of intraocular lenses, e.g. monofocal, bifocal, multifocal or EDOF lenses. In order to carry out quick testing of a large number of different intraocular lenses, the support can be designed as an exchangeable cuvette in which the IOL is arranged.

In summary, with the aid of the device according to the invention, the patient can, even before the implantation of an IOL, gain a realistic subjective visual impression—as though the IOL were implanted—over a larger field of view and the selection of a suitable IOL is facilitated.

In a preferred embodiment, at least one of the lens surfaces of the diverging and collecting lenses facing one another is an asphere. In the interaction with the concave-convex correcting lens and the corneal optic, a particularly good viewing angle correction, i.e. a particularly well focused view agreeing in resolution and contrast with the implanted IOL for different viewing angles, can thus be achieved through the IOL.

In an advantageous embodiment, at least one of the lens surfaces of the diverging and collecting lenses facing one another is an asphere at least of the fourth order. The use of such an aspherical lens permits optical aberrations, such as spherical aberrations, astigmatism, etc. to be prevented or corrected. The patient thus gains perfect vision over a wide field of view through the IOL and the precise testing thereof is thus enabled. Whilst the use of an asphere of higher order offers great flexibility in design of the ocular optic, an asphere of low order is easier to produce and its influence on the beam path is easier to predict.

In a particularly advantageous variant, the asphere of the fourth order and the arrow height measured away from the vertex plane of the asphere is selected according to

$${z}_{1} = \frac{ρ \cdot {r}^{2}}{1 + \sqrt{1 - {( ρ \cdot r )}^{2}}} + {A}_{2} \cdot {r}^{2} + {A}_{4} \cdot {r}^{4} with {A}_{4} < 0 , \qquad ( 1 )$$

with
z₁ . . . Arrow height, measured from distal to proximal,
ρ . . . Vertex curvature
r . . . Radial distance from the central viewing axis, and
A₂,A₄ . . . predefined coefficients, wherein A₂>0, when the asphere is a lens surface of a collecting lens, and A₂<0, when the asphere is a lens surface of a diverging lens.

Such a lens surface, the arrow height of which follows a constant function of low order, can be produced easily and precisely.

If the asphere is the distal lens surface of the collecting lens, the manufacture can be facilitated and the achievable viewing angle correction of the device can be further increased.

In order in particular to simplify the manufacture of the asphere, it is advantageous if the lens surface of the collecting or diverging lens lying opposite the asphere is planar. For this purpose, the collecting lens or the diverging lens can optionally consist of a plurality of individual lenses spaced apart from one another.

In a favourable variant, at least one of the corneal and ocular optics comprises an achromatic lens or an apochromatic lens in order to avoid chromatic aberrations. Lens tests, which agree with the implanted IOL in resolution, focusing and contrast over a wide field of view, can thus be carried out for a multiplicity of wavelengths. Furthermore, spherical aberrations can be prevented or corrected with an achromatic lens or apochromatic lens. In this variant, for example, a dispersion-free or incident angle-independent collimation of a beam of light passing through the device can be achieved in front of the patient's eye.

The corneal optic can be designed in different ways. In a variant which is particularly easy in terms of manufacture, the corneal optic comprises a corneal-diverging lens and a corneal-collecting lens, which can be arranged in any order along the optical viewing axis.

If the collecting and diverging lens of the corneal optic and/or those of the ocular optic have different Abbe numbers, the respective collecting and diverging lenses form an achromatic or apochromatic lens and, apart from the spherical aberrations, can also correct or eliminate chromatic aberrations. The patient is thus able to subject the IOL to a particularly careful examination and, even before its implantation, to judge the quality of the IOL both with regard to the acuity and also with regard to the colour perception and is thus able to find a suitable IOL.

It has been shown in tests that it is advantageous if the distal lens surface of the corneal collecting lens has an asphere at least of the fourth order. In the interaction with at least one asphere and the correcting lens of the ocular optic, a viewing angle-corrected lens test for a viewing angle of over 10° can thus be obtained for example, so that the patient is allowed a particularly realistic lens examination over a wide field of view.

It is particularly advantageous if the arrow height of this distal lens surface measured away from the vertex plane of the distal lens surface of the corneal collecting lens is selected according to

$${z}_{2} = \frac{ρ \cdot {r}^{2}}{1 + \sqrt{1 - {( ρ \cdot r )}^{2}}} + {A}_{2} \cdot {r}^{2} + {A}_{4} \cdot {r}^{4} with {A}_{2} > 0 and {A}_{4} < 0 , \qquad ( 2 )$$

with
z₂ . . . arrow height, measured from distal to proximal,
ρ . . . vertex curvature
r . . . radial distance from the central viewing axis and
A₂, A₄ . . . predefined coefficients.

Such a distal lens surface, the arrow height of which follows a constant function of low order, can be produced easily and precisely.

The correcting lens of the ocular optic can also be designed in a different ways. In a preferred embodiment, the distal and the proximal lens surface of the correcting lens are spheres and the radius of curvature of the distal lens surface of the correcting lens is smaller in size than the radius of curvature of the proximal lens surface of the correcting lens. With this correcting lens—in the interaction with the upstream corneal optic and the downstream diverging or collecting lenses of the ocular optic—a greatly extended field of view can be achieved while avoiding defocusing and contrast errors in the extended field of view.

In all the embodiments, it is advantageous if at least one of the elements corneal optic, mounting and ocular optic is mounted on the support displaceably along the central viewing axis. Each of the mentioned elements can either be displaced as a whole or, if one of the elements comprises sub-elements, e.g. the mentioned correcting, collecting and diverging lenses of the ocular optic, the sub-elements can also be displaceable separately. An adjustable mounting enables a rapid adaptation of the device, for example a displacement of the optical main plane of an optic, to the patient's eye that is just undergoing the vision test. The vergence of the light beam exiting proximally from the ocular optic can be adapted to the defective vision of the patient, for example for the correction of spherical refractive errors. By means of this adaptation, a lens test, which simulates the IOL in the implanted state with regard to contrast, resolution and focusing, can also be ensured for patients with defective eyesight.

It is also advantageous if at least one of the elements corneal optic, mounting and ocular optic are mounted displaceable on the support at right angles to the central viewing axis, in order to be able to adjust and calibrate the device easily.

Not least, it is advantageous if the mounting is designed to receive the intraocular lens exchangeably. Such a quick-change mounting allows an already tested IOL to be replaced in the device in a short time with a new IOL to be tested. As a result of the rapid exchange, the patient is able to efficiently test a large number of different IOLs and thus to select the IOL best suited to him on the basis of the subjective visual impression for a subsequent implantation.

In a further aspect of the invention, the use of an ocular optic, which comprises distally a correcting lens and, proximally thereto, a diverging lens and a collecting lens, wherein the correcting lens comprises a distal concave lens surface and a proximal convex lens surface, and wherein at least one of the lens surfaces of the diverging and collecting lenses facing one another is preferably an asphere, is provided in a device for testing the vision through an intraocular lens. With regard to the advantages and possible embodiments of the use of the ocular optic in an optical testing device, reference is made to the above comments concerning the device according to the invention.

The invention is explained in greater detail below with the aid of examples of embodiment shown in the appended drawings. In the figures:

FIG. 1 shows a device for testing the vision through an intraocular lens according to the prior art with an exemplary beam path for an eye looking in the central viewing axis onto the intraocular lens, in a diagrammatic side view;

FIG. 2 shows the device from FIG. 1 with an exemplary beam path for an eye looking at a viewing angle onto the intraocular lens, in a diagrammatic side view;

FIG. 3 shows the eye from FIG. 2 and a defocussed light beam cut off by the iris of the eye in the beam path from FIG. 2, in a diagrammatic side view;

FIG. 4 shows exemplary modulation transfer functions, such as are achieved for different viewing angles with the device of FIGS. 1 and 2, in a contrast-line diagram;

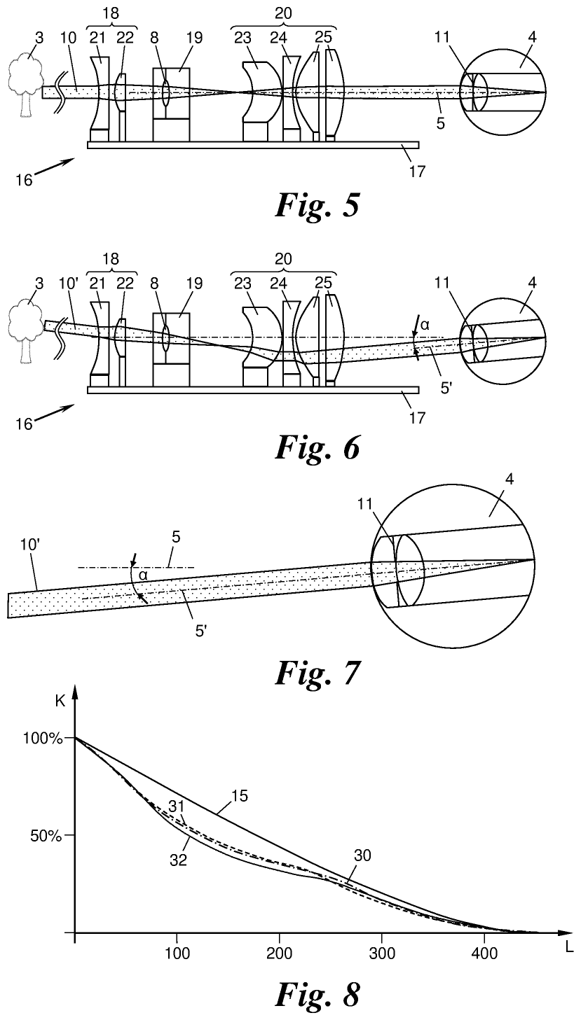

FIG. 5 shows a device for testing the vision through an intraocular lens according to the present invention with an exemplary beam path for an eye looking in the central viewing axis onto the intraocular lens, in a diagrammatic side view;

FIG. 6 shows the device from FIG. 5 with an exemplary beam path for an eye looking at a viewing angle onto the intraocular lens, in a diagrammatic side view;

FIG. 7 shows the eye from FIG. 6 and a focused light beam not cut off by the iris of the eye in the beam path from FIG. 6, a diagrammatic side view;

FIG. 8 shows exemplary modulation transfer functions, such as they are achieved for different viewing angles with the device of FIGS. 5 and 6, in a contrast-line diagram;

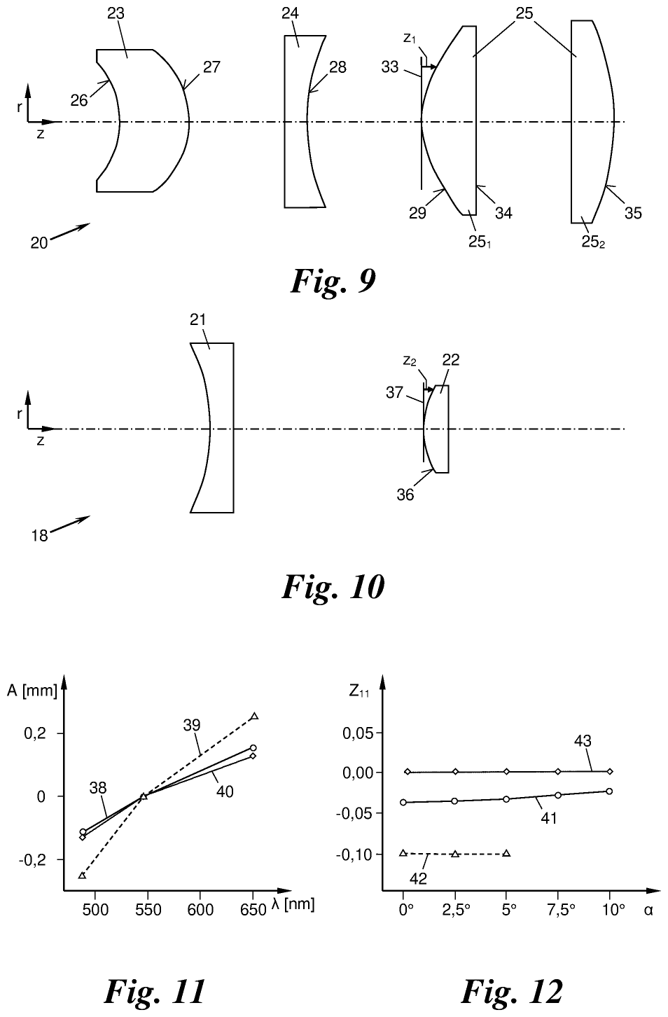

FIG. 9 shows the ocular optic of the device of FIGS. 5 and 6 in an exploded side view;

FIG. 10 shows the corneal optic of the device of FIGS. 5 and 6, in an exploded side view;

FIG. 11 shows exemplary chromatic aberrations, such as they occur in the device of FIG. 1 to 3 and in the device of FIG. 5 to 7, in a diagram of the longitudinal chromatic aberration over the wavelength; and

FIG. 12 shows exemplary spherical aberrations, such as they occur in the device of FIG. 1 to 3 or in the device of FIG. 5 to 7, in a diagram of Zernike coefficients over the viewing angle.

A conventional optical testing device 1 for an intraocular lens (IOL) 8, such as it is known in the prior art, has already been described above with the aid of FIG. 1 to 4. The reference numbers in FIG. 5 to 12 denote the same parts as in FIG. 1 to 4.

FIG. 5 to 7 show an inventive optical testing device 16 for testing vision through IOL 8 before its implantation into a patient's eye 4. IOL 8 to be tested, which after it implantation—referred to in the following as “implanted IOL”—is intended to lead to an improvement in the patient's vision, can for example be a monofocal, bifocal, multifocal, EDOF (extended depth of focus”) lens, a light adjustable lens (LAL), etc.

In order to give the patient a subjective visual impression through the not yet implanted IOL 8 onto a near, intermediate or distant environment 3, device 16 comprises a support 17, which supports a corneal optic 18, a mounting 19 for IOL 8 and an ocular optic 20 distally (close to the observed environment 3) to proximally (close to a patient's eye 4) along a central viewing axis. Support 17 can be for any device known in optics for mounting optical elements, e.g. a frame, a housing, a tube, an optical bank, an optical table, etc. Support 17 can support the optical elements either in a fixed position or displaceably. For example, corneal optic 18 (or parts thereof), mounting 19 (or parts thereof) and/or ocular optic 20 (or parts thereof) can be mounted displaceable along central viewing axis 5 and/or displaceable normal thereto on support 17, in order to adjust them relative to one another.

The optical effect of device 16 is to be described in the following with direct vision along central viewing axis 5 (FIG. 5) and with an oblique direct view along a viewing axis 5′ inclined at a viewing angle α (FIG. 6) with the aid of two representative light beams 10 (FIG. 5) and respectively 10′ (FIG. 6).

A light beam 10 or 10′ incident from the surroundings 3, i.e. from the distal end of device 16, first passes through corneal optic 18, which optically simulates a cornea, in the example shown through a distal corneal diverging lens 21 and a proximal corneal collecting lens 22. In a first variant, corneal optic 18 forms a cornea of an “average patient”, e.g. according to an eye model such as for example the Liou-Brennen eye model. In an alternative variant, corneal optic 18 simulates the cornea of eye 4 of an individual patient, for which purpose the latter is measured beforehand, e.g. optically or by means of ultrasound.

After corneal optic 18, light beam 10 or 10′ is guided through IOL 8 in mounting 19, where it focuses light beam 10 or 10′ according to its retractive power—as though it were implanted in eye 4.

Mounting 19 can be any mounting suitable for mounting IOL 8, e.g. a modular quick-change system with a first part fixedly mounted on support 17 for mounting a quickly exchangeable second part, in or on which IOL 8 is mounted in order to be able to quickly test IOLs by exchanging the second part. For example, mounting 19 can as the first part comprise a clamping, plug-in or magnetic mounting, which accommodates a replaceable cuvette for the IOL, optionally filled with a solution to simulate the eye interior, as the second part.

Next, light beam 10 or 10′ crosses through ocular optic 20, which comprises a distal correcting lens 23 and, proximal thereto, a diverging lens 24 and a collecting lens 25. As an alternative to the shown embodiments, collecting lens 25 can be arranged distally to diverging lens 24. Correcting lens 23 has a distal concave lens surface 26 and a proximal convex lens surface 27, see FIG. 9. Optionally, at least one or both of lens surfaces 28, 29 of diverging and collecting lenses 24, 25 facing one another is an asphere (here: distal lens surface 29 of collecting lens 25).

Through these special correcting, diverging and collecting lenses 23-25, light beams 10 and 10′ are changed in the ocular optic 20 depending on viewing angle α in vergence and direction, in such a way that, after passing through ocular optic 20, they are not cut off by iris 11 of eye 4 both in the case of a straight (FIG. 5: α=0°) and also in the case of an inclined viewing axis (FIGS. 6 and 7: α≠0°). In the case of a fixed viewing angle α, even light beams incident at slightly different angles are not of course cut off by iris 11, after which they are also focused on peripheral points of the retina of eye 4.

As can be seen from a comparison of FIGS. 3 and 7, through the interaction of correcting, diverging and collecting lenses 23-25 of ocular optic 20, light beam 10′ propagated along the inclined viewing axis 5′ in device 16 is no longer cut off by iris 11 of eye 4 and is correctly focused on the retina of eye 4. Consequently, the image perceived at a viewing angle α>0° is not only not cut off, but also is in better agreement in contrast and resolution with implanted IOL 8 than that of conventional device 1, as emerges from a comparison of the modulation transfer functions of device 1 shown in FIG. 4 with the modulation transfer functions of device 16 shown in FIG. 8 for α=0° (dot-dashed line 30), α=2.5° (dashed line 31) and α=5° (dotted line 32), in each case with the nominal MTF of implanted IOL 8 (continuous line 15), in diagrams of contrast K over line pairs L. In device 16 according to the invention, therefore, a larger field of view of IOL 8 can be tested than with conventional device 1.

In the embodiment of ocular optic 20 shown in FIGS. 5, 6 and 9, the at least one asphere of diverging and collecting lenses 24, 25 is formed by distal lens surface 29 of collecting lens 25. Aspheres can generally be described according to DIN ISO 10110 by the arrow height z measured away from the vertex plane of the lens surface as a function of radial distance r to central viewing axis 5 according to

$$z = \frac{ρ \cdot {r}^{2}}{1 + \sqrt{1 - ( 1 + k ) \cdot {( ρ \cdot r )}^{2}}} + {\sum }_{i = 1}^{I} {A}_{i} \cdot {r}^{i} \qquad ( 3 )$$

with
ρ . . . vertex curvature,
k . . . conical constant, and
Aᵢ . . . predefined coefficients.

In a variant of aspherical distal lens surface 29, this is an asphere at least of the fourth order, i.e. at least a coefficient Aᵢ with i≥4 in equation (3) is not equal to zero, wherein the first summand can either be present or not. In an exemplary sub-variant thereof, arrow height z₁ of distal lens surface 29 measured away from vertex plane 33 of distal lens surface 29 is selected according to

$${z}_{1} = \frac{ρ \cdot {r}^{2}}{1 + \sqrt{1 - {( ρ \cdot r )}^{2}}} + {A}_{2} \cdot {r}^{2} + {A}_{4} \cdot {r}^{4} with {A}_{2} > 0 and {A}_{4} < 0 , \qquad ( 1 )$$

with
z₁ . . . arrow height of distal lens surface 29, measured from distal to proximal,
ρ . . . vertex curvature,
r . . . radial distance from central viewing axis 5, und
A₂, A₄ . . . predefined coefficients.

In an alternative variant of ocular optic 20, in which its asphere is formed by proximal lens surface 28 of diverging lens 24, this lens surface 28 is an asphere at least of the fourth order, and in an exemplary sub-variant thereof the arrow height of the distal lens surface measured away from the vertex plane of proximal lens surface 28 is selected according to

$${z}_{{1}^{′}} = \frac{ρ \cdot {r}^{2}}{1 + \sqrt{1 - {( ρ \cdot r )}^{2}}} + {A}_{2} \cdot {r}^{2} + {A}_{4} \cdot {r}^{4} with {A}_{2} < 0 and {A}_{4} < 0 , \qquad ( {1}^{′} )$$

with
z_{1′} . . . arrow height of proximal lens surface 28, measured from distal to proximal,
ρ . . . vertex curvature,
r . . . radial distance from central viewing axis 5, and
A₂, A₄ . . . predefined coefficients.

In the embodiment shown, diverging lens 24 is optionally formed planar-concave and collecting lens 25 is formed from a first convex-planar segment 25₁ and a second planar-convex segment 25₂. Lens surface 34 lying opposite aspherical lens surface 29 is thus planar and the asphere is easier to produce. Furthermore, as a result of the curvature of the most proximal lens surface (here: proximal lens surface 35 of proximal segment 25₂), light beam 10 or 10′ is directed onto eye 4 collimated.

In a further optional embodiment, collecting and diverging lenses 24 and 25 can have different Abbe numbers in order to form an achromatic lens.

For the viewing angle enlargement, concave-convex correcting lens 23 can be designed in many ways, e.g. their distal and proximal lens surfaces 26, 27 can each in themselves be spherical or aspherical. In the variant shown in FIG. 9, the two lens surfaces 26, 27 of correcting lens 23 are spheres. Moreover, distal lens surface 26 has a greater curvature than proximal lens surface 27, i.e. the radius of curvature of the former is smaller in size than the radius of curvature of the latter.

Corneal optic 18 can also be designed in many ways. In the embodiment shown in FIGS. 5, 6 and 10, corneal optic 18 is formed by a (here: concave-planar) corneal diverging lens 21 and a (here: convex-planar) corneal collecting lens 22, which can be arranged either in the represented sequence with distal corneal diverging and proximal corneal collecting lens 21, 22 or the reverse sequence with distal corneal collecting and proximal corneal diverging lens 21, 22.

In a variant thereof, corneal diverging lens 21 and corneal collecting lens 22 have different Abbe numbers and thus form an achromatic lens, which corrects chromatic aberrations, optionally in addition to spherical aberrations.

Furthermore, in the embodiment shown, distal lens surface 36 of corneal collecting lens 22 is an asphere. In a variant, this asphere is of the fourth order, and in an exemplary sub-variant thereof arrow height z₂ of distal lens surface 36 measured away from vertex plane 37 of distal lens surface 36 is selected according to

$${z}_{2} = \frac{ρ \cdot {r}^{2}}{1 + \sqrt{1 - {( ρ \cdot r )}^{2}}} + {A}_{2} \cdot {r}^{2} + {A}_{4} \cdot {r}^{4} with {A}_{2} > 0 and {A}_{4} < 0 , \qquad ( 2 )$$

with
z₂ . . . arrow height, measured from distal to proximal,
ρ . . . vertex curvature,
r . . . radial distance from central viewing axis 5, and
A₂, A₄ . . . predefined coefficients.

Optionally or alternatively, corneal diverging lens 21 could comprise an aspherical lens surface.

Other embodiments of corneal optic 18 and/or ocular optic 20 with further or different optical elements are of course also possible, e.g. with an achromate or apochromate for colour correction and/or a proximal lens for focusing or defocusing, with mirrors for beam deflection, etc.

Furthermore, it should be stated that each of lenses 21—25 can be formed both in one piece and also as a plurality of segments—optionally be separated from one another, as shown here in the case of lens 25.

In tests, particularly good field of view extensions were able to be achieved with an aspherical lens surface 36 of corneal collecting lens 22 with a vertex curvature ρ between 0.058/mm and 0.118/mm, preferably 0.088/8 mm, a coefficient A₂ between 1·10⁻⁵/mm and 4.4·10⁻⁵/mm, preferably 2.7·10⁻⁵/mm, a coefficient A₄ between −0.6·10⁻⁴/mm³ and −1.6·10⁻⁴/mm³, preferably −1.1·10⁻⁴/mm³; and with an aspherical lens surface 29 of collecting lens 25 with an mit vertex curvature p between 0.01/mm and 0.09/mm, preferably 0.05/mm, a coefficient A₂ between 0.005/mm and 0.025/mm, preferably 0.015/mm, a coefficient A₄ between −1·10⁻⁵/mm³ and −9·10⁻⁵/mm³, preferably −5·10⁻⁵/mm³; and with a correcting lens 23 with a spherical distal lens surface 26 with a radius of curvature between −18 mm and −7 mm, preferably −12.5 mm, and with a spherical proximal lens surface 27 with a radius of curvature between −19 mm and −7 mm, preferably −13 mm.

FIGS. 11 and 12 illustrate the improvement achieved in tests with device 16 compared to conventional device 1 in respect of the longitudinal chromatic aberration (here: focus variation A over wavelength λ) along central viewing axis 5 (FIG. 11) and the spherical aberration on the basis of the Zernike coefficient Z₁₁ with a wavelength of 546 nm for different viewing angles α (FIG. 12). The continuous lines with circles 38 and 41 correspond to the results of device 16 according to the invention, the dashed lines with triangles 39 and 42 respectively to conventional device 1 and the dotted lines with rhombuses 40 and 43 respectively to the results of implanted IOL 8.

If desired, additional optical elements can also be arranged along central viewing axis 5, for example to correct aberrations or to extend possible viewing angles α for testing IOL 8. Furthermore, device 16 can alternatively be designed completely or partially with non-rotation-symmetrical lenses, for example the aspherical lens surfaces of the corneal optic 18 and/or the ocular optic 20 described here can be replaced by systems comprising a plurality of spherical lens surfaces, which achieve a comparable optical effect.

Not least, two IOLs can of course be tested simultaneously and in binocular (stereoscopic) sight with a setup comprising two parallel devices 16.

Very generally, ocular optic 20, optionally combined with corneal optic 18, can be used in any device for testing vision through an IOL 8.

The invention is not limited to the represented embodiments, but also includes all variants, modifications and combinations that fall within the scope of the appended claims.

1. A device for testing vision through an intraocular lens (8), with a support (17) which supports a corneal optic (18), a mounting (19) for the intraocular lens (8) and an ocular optic (20) one after the other distally to proximally in a central viewing axis (5),
wherein the ocular optic (20) comprises distally a correcting lens (23) and, proximally thereto, a diverging lens (24) and a collecting lens (25),
wherein the correcting lens (23) comprises a distal concave lens surface (26) and a proximal convex lens surface (27).

2. The device according to claim 1, wherein at least one of the lens surfaces (28, 29) of the diverging and collecting lenses (24, 25) facing one another is an asphere.

3. The device according to claim 2, wherein at least one of the lens surfaces (28, 29) of the diverging and collecting lenses (24, 25) facing one another is an asphere at least of the fourth order.

4. The device according to claim 3, wherein the arrow height (z₁) measured from the vertex plane (33) of the asphere is selected according to
$${z}_{1} = \frac{ρ \cdot {r}^{2}}{1 + \sqrt{1 - {( ρ \cdot r )}^{2}}} + {A}_{2} \cdot {r}^{2} + {A}_{4} \cdot {r}^{4} with {A}_{4} < 0 ,$$
with
zᵢ . . . Arrow height, measured from distal to proximal,
ρ . . . Vertex curvature
r . . . Radial distance from the central viewing axis (5), and
A₂,A₄ . . . predefined coefficients, wherein A₂>0, when the asphere is a lens surface of a collecting lens (24), and A₂<0, when the asphere is a lens surface of a diverging lens (25).

5. The device according to claim 2, wherein the asphere is the distal lens surface (29) of the collecting lens (25).

6. The device according to claim 2, wherein the lens surface (34) of the collecting or diverging lens (25, 24) lying opposite the asphere is planar.

7. The device according to claim 1, wherein at least one of the corneal and ocular optics (18, 20) comprises an achromatic lens (21, 22; 24, 25) or an apochromatic lens.

8. The device according to claim 1, wherein the corneal optic (18) comprises a corneal diverging lens (21) and a corneal collecting lens (22).

9. The device according to claim 7, wherein the corneal diverging lens (21) and the corneal collecting lens (22) have different Abbe numbers and/or the collecting and diverging lenses (24, 25) of the ocular optic (20) have different Abbe numbers.

10. The device according to claim 9, wherein the distal lens surface (36) of the corneal collecting lens (22) is an asphere at least of the fourth order.

11. The device according to claim 10, wherein the arrow height (z₂) of the distal lens surface measured away from the vertex plane (37) of the distal lens surface (36) of the corneal collecting lens (22) is selected according to
$${z}_{2} = \frac{ρ \cdot {r}^{2}}{1 + \sqrt{1 - {( ρ \cdot r )}^{2}}} + {A}_{2} \cdot {r}^{2} + {A}_{4} \cdot {r}^{4} with {A}_{2} > 0 and {A}_{4} < 0 ,$$
with
z₂ . . . Arrow height, measured from distal to proximal,
ρ . . . Vertex curvature
r . . . Radial distance from the central viewing axis (5), and
A₂,A₄ . . . predefined coefficients.

12. The device according to claim 1, wherein the distal and the proximal lens surface (26, 27) of the correcting lens (23) are spheres, wherein the radius of curvature of the distal lens surface (26) of the correcting lens (23) is smaller in size than the radius of curvature of the proximal lens surface (27) of the correcting lens (23).

13. The device according to claim 1, wherein at least one of the elements corneal optic (18), mounting (19) and ocular optic (20) are mounted displaceably on the support (17) along the central viewing axis (5) and/or at right angles to the central viewing axis (5).

14. The device according to claim 1, wherein the mounting (19) is designed to receive the intraocular lens (8) exchangeably.

15. Use of an ocular optic (20), which comprises distally a correcting lens (23) and, proximally thereto, a diverging lens (24) and a collecting lens (25), wherein the correcting lens (23) comprises a distal concave lens surface (26) and a proximal convex lens surface (27), in a device (16) for testing vision through an intraocular lens (8).

16. The use according to claim 15, and wherein at least one of the lens surfaces (28, 29) of the diverging and collecting lenses (24, 25) facing one another is an asphere.
